(19) United States
(12) Patent Application Publication
(10) Pub. No.: US 2022/0233623 A1
(43) Pub. Date: Jul. 28, 2022

(54) COMPOSITION, COMPRISING AUCKLANDIA LAPPA DECNE. EXTRACT, FOR PREVENTING HAIR LOSS OR PROMOTING HAIR REGROWTH

(71) Applicant: OKCHUNDANG CO., LTD., Daegu (KR)

(72) Inventors: Chul Jong JUNG, Daegu (KR); Gyung Yeun BEIK, Daegu (KR)

(21) Appl. No.: 17/572,943

(22) Filed: Jan. 11, 2022

(30) Foreign Application Priority Data
Jan. 22, 2021 (KR) 10-2021-0009494

(51) Int. Cl. A61K 36/285; A61P 17/14

(52) U.S. Cl. CPC A61K 36/285; A61P 17/14; A61K 2236/35; A61K 2236/333; A61K 2236/331

(57) ABSTRACT

Disclosed herein are a composition comprising an Aucklandia lappa Decne. extract, especially a crude solvent extract from Aucklandia lappa as an active ingredient for alleviation, inhibition, prevention, or treatment of hair loss, and a preparation method therefor. The Aucklandia lappa Decne. extract can be used in a pharmaceutical or food composition useful for alleviating, inhibiting, preventing, and treating hair loss.

### BACKGROUND OF THE INVENTION

### Field of the Invention

This application claims priority to and the benefit of Korean Patent Application Nos. 10-2021-0009494 filed 22 Jan., 2021 in the Korean Intellectual Property Office, the content of which is incorporated herein in its entirety by reference.

The present disclosure relates to a composition comprising an Aucklandia lappa Decne. extract, especially a crude solvent extract from Aucklandia lappa as an active ingredient for alleviation, inhibition, prevention, or treatment of hair loss, and a preparation method therefor. The Aucklandia lappa Decne. extract according to the present disclosure can be used in a pharmaceutical or food composition useful for alleviating, inhibiting, preventing, and treating hair loss.

The present disclosure relates to a method for prevention or treatment of hair loss, the method comprising:

administering a composition comprising an Aucklandia lappa Decne. extract as an active ingredient to a subject in need thereof.

The present disclosure relates to a method for alleviation or inhibition of hair loss, the method comprising:

administering a composition comprising an Aucklandia lappa Decne. extract as an active ingredient to a subject in need thereof.

The present disclosure relates to a method for promotion of hair regrowth, the method comprising:

administering a composition comprising an Aucklandia lappa Decne. extract as an active ingredient to a subject in need thereof.

### Description of the Prior Art

With the increase of interest in appearance, hair loss symptoms have recently become a big concern. Hair loss was usually considered only a concern for men in their 40s and 50s, but now the number of hair loss patients in their 20s and 30s is soaring, and women's hair loss is also increasing. These hair loss symptoms are becoming worse due to many environmental factors such as dietary imbalance, harmful air, contaminated drinking water, acid rain, and stress in modern people. Accordingly, many studies are underway to prevent and treat hair loss symptoms.

The hair growth cycle includes the following three stages: anagen, which is the growth phase of hairs; catagen, which signals the end of the active growth of a hair with the regression of the hair bulb; telogen, which is the resting phase where the hair papilla stops working with the hair remaining in the scalp, and exogen in which the hair papilla begins to work or a new hair is generated while an old hair is shedding.

Anagen Stage (2-7 years) is the phase during which hair grows and is divided into the stage of hair generation in which hair is about to go out from the hair bulb to the hair follicle, and the stage in which hard keratin is made inside the hair follicle. Hairs continue to grow by themselves until the catagen stage.

Catagen Stage (2-3 weeks) is a time when the metabolic process slows down while maintaining the shape of hair after the growth period, and keratin is not produced at this stage. Normally, about 1% of hair follicles are in catagen. The hair bulb shrinks to separate from the hair papilla and goes upward while being surrounded by the hair follicle. In this stage, the cell division is halted.

Telogen Stage (3 months) is a phase which lasts 3-4 months until the next anagen stage and in which the hair the hair papilla shrinks and the hair follicle gradually dwindles while the hair root is pushed upward and falls out.

At any point in time, most of a person's total amount of hair is in the anagen stage. In persons suffering from alopecia, an increased number of hairs are in the telogen stage, compared to normal persons, so that remaining hairs become fewer in number, leading to an appearance of baldness. With the advance of hair loss, the period of the anagen stage become short, which results in gradual shortening of the hairs. For treatment of hair loss, therefore, it is important to switch hair follicles from the telogen stage to the anagen stage within a short period and to prolong the shortened period of the anagen stage.

There are two types of hair regrowth promoting agents approved by the FDA: minoxidil (topical use) and finasteride (oral medication). Minoxidil was used as an oral medicine for hypertension, but patients who had taken the medicine were observed to exhibit hypertrichosis. Based on this side effect, minoxidil is currently used as a topical medication for male pattern baldness. Minoxidil is a pyrimidine derivative that widens blood vessels in the scalp to allow more blood to hair follicles and activate germinal matrix cells, resulting in delay of hair loss and growth of downy hairs. Finasteride is the first oral medication for androgenic alopecia, which inhibits type II 5α-reductase to prevent hair loss and promote hair regrowth. Since the approval thereof by the FDA in 1997, about 2.6 million persons are currently prescribed the medication.

However, adverse effects of minoxidil have been reported to include weight gain, edema, increased heartbeat, angina, dermatitis, and itching. For finasteride, clinical cases such as sexual dysfunction, etc. are reported as adverse effects. Thus, there are the problems that the medications are limitative in application or patients themselves show reluctance to the medications.

Accordingly, there is an increase in consumer interest in safe substances, derived from natural materials, for hair loss prevention and hair regrowth promotion, and research on this is also being actively conducted.

### SUMMARY OF THE INVENTION

Leading to the present disclosure, intensive and through research, conducted by the present inventors, into the development of an agent for alleviating, preventing, or treating hair loss, resulted in the finding that of more than 50 medicinal herbs and folk medicines screened, Aucklandia lappa promotes the growth and proliferation of hair growth cells with significance and ultimately exhibits a hair regrowth effect.

Accordingly, an aspect of the present disclosure is to provide a pharmaceutical composition comprising an Aucklandia lappa Decne. extract as an active ingredient for alleviation, prevention, or treatment of hair loss.

Another aspect of the present disclosure is to provide a food composition comprising an Aucklandia lappa Decne. extract as an active ingredient for alleviation or inhibition of hair loss.

A further aspect of the present disclosure is to provide a food composition comprising an Aucklandia lappa Decne. extract as an active ingredient for promotion of hair regrowth.

A still further aspect of the present disclosure is to provide a method for alleviating, prevention, or treating hair loss, the method comprising administering an Aucklandia lappa Decne. extract in an amelioratively, prophylactically, or therapeutically effective amount to a subject in need thereof.

Still another aspect of the present disclosure is to provide a use of an Aucklandia lappa Decne. extract for alleviation, prevention, or treatment of hair loss.

Yet another aspect of the present disclosure is to provide a method for preventing or treating hair loss, the method comprising administering a composition comprising an Aucklandia lappa Decne. extract as an active ingredient to a subject in need thereof.

A yet further aspect of the present disclosure is to provide a method for alleviating or inhibiting hair loss, the method comprising administering a composition comprising Aucklandia lappa Decne. extract as an active ingredient to a subject in need thereof.

Yet still another aspect of the present disclosure is to provide a method for promoting hair regrowth, the method comprising administering a composition comprising an Aucklandia lappa Decne. extract as an active ingredient to a subject in need thereof.

### BRIEF DESCRIPTION OF THE DRAWINGS

The above and other aspects, features and advantages of the present disclosure will be more apparent from the following detailed description taken in conjunction with the accompanying drawings, in which:

FIG. 1 shows microscopic images of stained liver and kidney tissues after oral administration of Aucklandia lappa crude extracts according to an embodiment;

FIG. 2A shows photographic images illustrating hair growth extents on shaved areas of mice after oral administration of Aucklandia lappa crude extracts according to an embodiment;

FIG. 2B is a graph of the quantitated ratios of hair growth on shaved areas after oral administration of Aucklandia lappa crude extracts according to an embodiment;

FIG. 3A shows microscopic images illustrating hair follicle states in the skin tissues stained after oral administration of Aucklandia lappa crude extracts according to an embodiment; and

FIG. 3B shows microscopic images illustrating distributions of fibrous tissues in the skin stained after oral administration of Aucklandia lappa crude extracts according to an embodiment.

### DETAILED DESCRIPTION OF THE EXEMPLARY EMBODIMENTS

The present disclosure pertains to a composition comprising an Aucklandia lappa Decne. extract, especially a crude solvent extract from Aucklandia lappa as an active ingredient for alleviation, inhibition, prevention, or treatment of hair loss, and a preparation method therefor.

Aucklandia lappa is a perennial dicotyledon belonging to the family Asteraceae in the order Asterales, native to Europe. The stall stands straight with a typical growth of 0.8-2.0 m tall. The plant has hairs densely grown thereacross and is cultivated as a medicinal herb. The leaves are alternate and take oval or long oval shapes, with serrated edges, and dense hairs on the back. Flowers blossom in July to August and are yellow with a diameter of 5 to 10 cm, and one capitate flower hangs on the leaf axil. The involucrum is hemispherical and 24 mm long, with an egg-shaped outer bract having dense hairs thereon as in leaves. The fruit is an achene with light reddish brown pappi. The root is used as a diaphoretic, a diuretic, and an expectorant and contains an anthelminthic ingredient.

The roots are cylindrical or semi-cylindrical and runs 5 to 10 cm, with a diameter of 0.5 to 5 cm. The root head is shaped like a dry bone and has a yellowish or grayish brown outside surface with distinct wrinkles, vertical grooves, and root marks. The roots are hard and do not break easily. The bent side is grayish or dark brown, and the surrounding area is grayish yellow or light yellowish brown. The rings of cambia are brown and have radial patterns with dot-shaped oil chambers scattered therein. The roots have a unique small and taste somewhat bitter.

Medicinally active ingredients including sesquiterpene and amino acids (sesquiterpene conjugate-saussureamine A-E, shikokiol, α-, β-ionone, phell andrene, camphene) are found in Aucklandia lappa and exhibit various physiological activities including aromatic stomachic, antiulcer, antibacterial, antiprotozoal, anti-inflammatory, and anti-cancer effects. An extract from the plant including costunolide was reported to have the pharmaceutical activity of promoting the release of bile juice and preventing stress-induced stomach ulcer. An anti-ulcer effect was also observed in the amino acid-sesquiterpene conjugate. This conjugate has been studied for anticancer activity such as angiogenesis inhibition, apoptotic induction, and inhibitory activity against the expression of iNOS and heat shock protein 72. Studies have also been conducted into the cytotoxicity of sesquiterpenen lactone compounds such as costunolide on cancer cells. Besides, anti-inflammatory activity, antibacterial activity, anthelmintic activity, anti-HBV activity, antihypertensive activity, and actions on the cardiovascular system and respiratory system were reported.

The present disclosure relates to a composition, comprising an Aucklandia lappa Decne. extract, for alleviation, inhibition, prevention, or treatment of hair loss. The term “extract” is intended to encompass a crude solvent extract, and the Aucklandia lappa Decne. extract may be in a solution, a concentrate, or a powder state.

In the present disclosure, the Aucklandia lappa may be at least one entity selected from the group consisting of roots, stems, and leaves thereof, for example, a root entity, but with no limitations thereto.

In the present disclosure, the Aucklandia lappa Decne. extract may be an extract obtained using at least one solvent selected from the group consisting of water, a straight or branched alcohol of 1 to 4 carbon atoms, acetone, ethyl acetate, butyl acetate, 1,3-butylene glycol, hexane, and diethyl ether, but with no limitations thereto.

In the present disclosure, the Aucklandia lappa Decne. extract may be an extract obtained using water as a solvent. For example, the extract may be obtained using 100% water as a solvent, but with no limitations thereto.

In the present disclosure, a mixture of water and alcohol may be used as a solvent for obtaining a crude extract from Aucklandia lappa. In this regard, the mixture may be a 10% (v/v) (inclusive) to 100% (v/v) (exclusive), 20% (v/v) (inclusive) to 100% (v/v) (exclusive), 30% (v/v) (inclusive) to 100% (v/v) (exclusive), 40% (v/v) (inclusive) to 100% (v/v) (exclusive), 50% (v/v) (inclusive) to 100% (v/v) (exclusive), 60% (v/v) (inclusive) to 100% (v/v) (exclusive), 10% (v/v) (inclusive) to 90% (v/v) (exclusive), 10% (v/v) (inclusive) to 80% (v/v) (exclusive), 10% (v/v) (inclusive) to 70% (v/v) (exclusive), 10% (v/v) (inclusive) to 60% (v/v) (exclusive), 10% (v/v) (inclusive) to 50% (v/v) (exclusive), 10% (v/v) (inclusive) to 40% (v/v) (exclusive), 10% (v/v) (inclusive) to 35% (v/v) (exclusive), 20% (v/v) (inclusive) to 90% (v/v) (exclusive), 20% (v/v) (inclusive) to 80% (v/v) (exclusive), 20% (v/v) (inclusive) to 70% (v/v) (exclusive), 20% (v/v) (inclusive) to 60% (v/v) (exclusive), 20% (v/v) (inclusive) to 50% (v/v) (exclusive), 20% (v/v) (inclusive) to 40% (v/v) (exclusive), 20% (v/v) (inclusive) to 35% (v/v) (exclusive), for example, 30% (v/v) aqueous solution of an alcohol of 1 to 4 carbon atoms, but with no limitations thereto.

In the present disclosure, the aqueous alcohol solution may be at least one selected from the group consisting of an aqueous methanol solution, an aqueous ethanol solution, an aqueous isopropanol solution, an aqueous propanol solution, and an aqueous butanol solution, but is not limited thereto.

Another aspect of the present disclosure pertains to a method for preparing an Aucklandia lappa Decne. extract having ameliorative, inhibitory, prophylactic, or therapeutic activity for hair loss.

In an embodiment of the present disclosure, the Aucklandia lappa may be at least one entity selected from the group consisting of roots, stems, and leaves and may be, for example, a root entity of the plant, but with no limitations thereto.

In the present disclosure, the Aucklandia lappa Decne. extract may be an extract obtained using at least one solvent selected from the group consisting of water, a straight or branched alcohol of 1 to 4 carbon atoms, acetone, ethyl acetate, butyl acetate, 1,3-butylene glycol, hexane, and diethyl ether, but with no limitations thereto.

In the present disclosure, the Aucklandia lappa Decne. extract may be an extract obtained using water as a solvent. For example, the extract may be obtained using 100% water as a solvent, but with no limitations thereto.

The preparation method for an Aucklandia lappa Decne. extract according to the present disclosure is described in detail as follows:

a drying step of sectioning Aucklandia lappa, washing the sections with water to remove adulterations, and drying the same;

an extraction step of obtaining an extract from Aucklandia lappa with a solvent;

a concentration step of concentrating the extract in a vacuum to afford a concentrate; and

a drying step of freeze drying the concentrate to collect powder.

The extraction step is to isolate a useful ingredient from a liquid or solid material by solubilizing the same in a solvent and may take advantage of room temperature extraction, cold precipitation extraction, hot water extraction, ultrasonic wave extraction, vapor extraction, reflux condensation extraction, or vacuum or compression extraction, and preferably reflux extraction. For reflux extraction, a cooling device installed at an upper portion of an extractor cools and condenses the vaporized solvent to minimize the amount of the solvent vaporizing away from the liquid phase.

In the extraction step, an extract may be obtained using about 5- to 20-fold volumes, about 5 to 15 volumes, and preferably 10-fold volumes of a solvent relative to the weight of Aucklandia lappa.

The concentration step is to increase the concentration of a solid content by removing the liquid from a liquid-phase material having a high content of liquid. In the present disclosure, a vacuum concentration method is employed. Vacuum concentration is designed to enrich a solute as a result of rapid evaporation of a solvent by reducing the pressure to decrease the boiling point of the solvent.

The concentrate may be dried using hot air dry (AD), cold air dry, vacuum dry (VD), spray dry (SD), or freeze dry (FD). Preferably, the drying step may be conducted by freeze drying. Freeze drying is a process that involves freezing a sample, lowering pressure, and then removing the ice by sublimation. Causing minimal damage to heat-labile substances, the freeze drying is applied to substances that are apt to be inactivated and broken by heat. Freeze-dried substances can be rehydrated precisely, cleanly, rapidly, and completely.

The extraction may be conducted by any method that is typically used in the art. Examples of the extraction method include hot water extraction, cold precipitation extraction, reflux condensation extraction, solvent extraction, vapor distillation, ultrasonic extraction, elution, and compression, but are not limited thereto.

The extract obtained by the extraction method may be further subjected to a typical fraction process, but with no limitations thereto.

The extract obtained by the extraction method may be purified using a typical purification method.

In the present disclosure, the Aucklandia lappa Decne. extract may be produced as a powder through an additional process of vacuum distillation and freeze dry or spray dry for the extract obtained by the extraction method.

In the present disclosure, the Aucklandia lappa Decne. extract may be produced as a fraction purified by various chromatographic methods, such as silica gel column chromatography, thin layer chromatography, high-performance liquid chromatography, etc.

The pharmaceutical composition of the present disclosure may comprise s pharmaceutically effective amount of the Aucklandia lappa Decne. extract and optionally a pharmaceutically acceptable carrier.

As used herein, the term “pharmaceutically effective amount” refers to a sufficient amount to achieve the efficacy or activity of the Aucklandia lappa Decne. extract.

So long as it is typically used, any pharmaceutically acceptable carrier may be contained in the pharmaceutical composition of the present disclosure. Examples of the pharmaceutically acceptable carrier include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, and mineral oil, but are not limited thereto. In addition to this ingredients, the pharmaceutical composition of the present disclosure may further comprise a lubricant, a humectant, a sweetener, a flavorant, an emulsifier, a suspending agent, a preservative, etc.

The pharmaceutical composition of the present disclosure may be administered to mammals including humans via various routes. Any administration mode that is typically used may be available and, for example, the composition be administered orally, intradermally, intravenously, intramuscularly, subcutaneously, etc., with preference for oral administration.

The suitable dose of the pharmaceutical composition according to the present disclosure may vary depending on various factors including the formulation method, administration mode, the patient's age, body weight, sex, pathological condition, diet, administration time, administration route, excretion rate, and response sensitivity. Usually, a skilled practitioner can easily determine and prescribe doses effective for desired therapy or prophylaxis.

The composition of the present disclosure may further contain a pharmaceutically suitable and physiologically acceptable auxiliary agent such as a carrier, an excipient, a diluent, etc. in addition to the extract.

Examples of the carrier, excipient, and diluent contained in the composition of the present disclosure include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

For formulation, a general diluent or excipient such as a filler, an extender, a binder, a humectant, a disintegrant, a surfactant or the like may be used. As a solid formulation for oral administration, a tablet, a pill, a pulvis, a granule, a capsule, etc. may be used, and such a solid formulation may be prepared by mixing the extract with at least one excipient, for example, starch, calcium carbonate, sucrose, lactose, gelatin, etc. Also, in addition to the excipient, a lubricant, such as magnesium stearate and talc, may be used.

A formulation for oral administration is exemplified by a suspension, a liquid for internal use, an emulsion, a syrup, an ointment, or the like, and may contain various excipients, for example, a humectant, a sweetener, an aromatic, a preservative, and the like in addition to frequently used simple diluents, such as water, liquid paraffin, etc.

A formulation for parenteral administration or an external administration for oral cavity may comprise at least one of a sterile aqueous solution, a non-aqueous solvent, suspension, an emulsion, a freeze-dried preparation, and a transdermal formulation. As the non-aqueous solvent and the suspension, propylene glycol, polyethylene glycol, vegetable oil such as olive oil, injectable ester such as ethyl oleate or the like may be used.

Examples of a formulation for parenteral administration include sterilized aqueous solution, non-aqueous solvents, suspensions, emulsions, freeze-dried formulations, suppositories, and transcutaneous agents. Propylene glycol, polyethylene glycol, vegetable oil, such as olive oil, and injectable esters, such as ethyloleate, can be used as non-aqueous solvent or suspension.

The base for suppositories includes Witepsol, macrogol, tween 61, cacao oil, laurin oil, glycerol and gelatin.

In an embodiment where the composition of the present disclosure is applied to humans, the herb extract composition of the present disclosure may be administered alone or in mixture with selected pharmaceutical vehicles in consideration of typical administration modes and the standard pharmaceutical practice.

For examples, the pharmaceutical composition of the present disclosure can be dosed orally, buccally or sublingually in the form of tablets containing starch or lactose, capsules alone or with excipients, or elixir or suspension with chemicals for taste masking or coloration. Such liquid preparations can be formulated with pharmaceutically acceptable additives such as suspending agents (e.g., methyl cellulose, semisynthetic glyceride such as Witepsol, a mixture of apricot kernel oil and PEG-6 ester, or a glyceride mixture such as PEG-8 and caprylic/capric glyceride).

The administration dose of the pharmaceutical composition comprising an Aucklandia lappa Decne. extract according to the present disclosure may vary depending on the patient's age, weight, and sex, administration mode, health status, and disease severity. In addition, the composition can be administered in divided doses once to several times a day at fixed time intervals according to the decision of a physician or pharmacist. For example, the daily dose may be 0.001 to 2 g/kg, preferably 0.5 to 500 mg/kg, based on the content of the active ingredient. The above doses are exemplified as an average case, and its dose may increase or decrease depending on individual differences.

If the daily dose of the composition containing the Aucklandia lappa Decne. extract of the present disclosure is less than the lower limit of the range, a significant effect cannot be obtained. A daily dose exceeding the upper limit of the range is not economically beneficial. In addition, undesirable side effects may be brought about as the dose deviates from usual ranges.

Another aspect of the present disclosure pertains to a food composition comprising the Aucklandia lappa Decne. extract as an active ingredient for alleviation or inhibition of hair loss.

In the present disclosure, the Aucklandia lappa may be at least one entity selected from the group consisting of roots, stems, and leaves thereof, for example, a root entity, but with no limitations thereto.

In the present disclosure, the Aucklandia lappa Decne. extract may be an extract obtained using at least one solvent selected from the group consisting of water, a straight or branched alcohol of 1 to 4 carbon atoms, acetone, ethyl acetate, butyl acetate, 1,3-butylene glycol, hexane, and diethyl ether, but with no limitations thereto.

In the present disclosure, the Aucklandia lappa Decne. extract may be an extract obtained using water as a solvent. For example, the extract may be obtained using 100% water as a solvent, but with no limitations thereto.

Another aspect of the present disclosure pertains to a food composition comprising an Aucklandia lappa Decne. extract as an active ingredient for promotion of hair regrowth.

In the present disclosure, the Aucklandia lappa may be at least one entity selected from the group consisting of roots, stems, and leaves thereof, for example, a root entity, but with no limitations thereto.

In the present disclosure, the Aucklandia lappa Decne. extract may be an extract obtained using at least one solvent selected from the group consisting of water, a straight or branched alcohol of 1 to 4 carbon atoms, acetone, ethyl acetate, butyl acetate, 1,3-butylene glycol, hexane, and diethyl ether, but with no limitations thereto.

In the present disclosure, the Aucklandia lappa Decne. extract may be an extract obtained using water as a solvent. For example, the extract may be obtained using 100% water as a solvent, but with no limitations thereto.

In the present disclosure, a mixture of water and alcohol may be used as a solvent for obtaining a crude extract from Aucklandia lappa. In this regard, the mixture may be a 10% (v/v) (inclusive) to 100% (v/v) (exclusive), 20% (v/v) (inclusive) to 100% (v/v) (exclusive), 30% (v/v) (inclusive) to 100% (v/v) (exclusive), 40% (v/v) (inclusive) to 100% (v/v) (exclusive), 50% (v/v) (inclusive) to 100% (v/v) (exclusive), 60% (v/v) (inclusive) to 100% (v/v) (exclusive), 10% (v/v) (inclusive) to 90% (v/v) (exclusive), 10% (v/v) (inclusive) to 80% (v/v) (exclusive), 10% (v/v) (inclusive) to 70% (v/v) (exclusive), 10% (v/v) (inclusive) to 60% (v/v) (exclusive), 10% (v/v) (inclusive) to 50% (v/v) (exclusive), 10% (v/v) (inclusive) to 40% (v/v) (exclusive), 10% (v/v) (inclusive) to 35% (v/v) (exclusive), 20% (v/v) (inclusive) to 90% (v/v) (exclusive), 20% (v/v) (inclusive) to 80% (v/v) (exclusive), 20% (v/v) (inclusive) to 70% (v/v) (exclusive), 20% (v/v) (inclusive) to 60% (v/v) (exclusive), 20% (v/v) (inclusive) to 50% (v/v) (exclusive), 20% (v/v) (inclusive) to 40% (v/v) (exclusive), 20% (v/v) (inclusive) to 35% (v/v) (exclusive), for example, 30% (v/v) aqueous solution of an alcohol of 1 to 4 carbon atoms, but with no limitations thereto.

The content of the Aucklandia lappa Decne. extract as an active ingredient contained in the food composition of the present disclosure is not particularly limited by the type of the food, the desired use, etc., and for example, it may be added in an amount of 0.01 to 15% by weight, based on the total weight of the food, and also added in an amount of 0.02 to 10 g, preferably 0.3 to 1 g, based on 100 ml of a health drink composition.

No particular limitations are imparted to types of the food. Examples of the food to which the material may be added include meats, sausage, bread, chocolate, candies, snacks, confectionery, pizza, ramen, other noodles, chewing gums, dairy products including ice creams, various soups, beverages, teas, drinks, alcoholic drinks, and vitamin complexes. As used herein, the term “food” is intended to encompass all foods in typical senses.

In the beverage, various flavoring agents or natural carbohydrates may be contained as additional ingredients. The carbohydrates may be natural sweeteners including monosaccharides, such as glucose, fructose, etc., disaccharides, such as maltose, sucrose, etc., and polysaccharides, such as dextrin, cyclodextrin, etc., or synthetic flavoring agents such as saccharin, aspartame, etc. The ratio of the natural carbohydrate may be determined according to the selection of a person skilled in the art.

Besides, the food composition of the present disclosure may contain various nutrients, vitamins, minerals (electrolyte), flavorants, colorants, pectic acid and salts thereof, alginic acid and salts thereof, organic acids, protective colloidal thickeners, pH adjusting agents, stabilizers, preservatives, glycerin, alcohols, carbonizing agents as used in carbonated beverages and the like. Moreover, the food composition of the present disclosure may contain fruit flesh, as used in preparing natural fruit juices and fruit juice beverages and vegetable beverages. These components can be used independently or in combination. The proportion of these additives is generally selected from a range of 0 to about 20% by weight based on the total weight of the composition of the present disclosure.

In the present disclosure, the food composition may be prepared and processed into a form of a tablet, a capsule, a pulvis, a granule, a liquid, a pill, and so on.

In the present disclosure, the food composition may be a “health functional food composition”. In this regard, the health functional food composition refers to a food prepared or processed with a raw material or ingredient having useful functionality for the human body according to the Health Functional Food Act No. 6727. The health functional food composition means a food that is consumed to control nutrients for the structure and function of the human body or to acquire useful effects for health purposes such as physiological actions.

The health functional food of the present disclosure may comprise conventional food additives and, unless otherwise specified, suitability as food additives is determined by standard and criteria on corresponding product according to the General Rules and General Test for Korean Food Additives Codex approved by the Korean Ministry of Food and Drug Safety. Examples of the items listed in the above-mentioned “Food Additives Codex” include chemical compounds such as ketones, glycine, calcium citrate, nicotinic acid, and cinnamic acid; natural additives such as persimmon color, licorice extract, crystalline cellulose, kaoliang color, and guar gum; mixture preparations such as L-glutamic acid sodium preparations, alkali agents for noodles, preservative preparations, and tar coloring preparations. For the health functional food in a tablet form, by way of example, the active ingredient of the present disclosure is mixed with an excipient, a binder, a disintegrant, and other additives, and granulated in a usual manner, followed by compression molding of the mixture together with a lubricant or by direct compression molding of the mixture. In addition, the health functional food in the tablet form may contain a flavoring agent or the like as needed. The hard capsule of the capsule-type health functional food can be prepared by filling a conventional hard capsule with a mixture of the active ingredient of the present disclosure and an additive such as an excipient. The soft capsule may be prepared by filling a capsule base such as gelatin with a mixture of the active ingredient of the present disclosure and an additive such as an excipient. The soft capsule may contain plasticizers such as glycerin and sorbitol, coloring agents, preservatives and the like, if necessary. The pill-type health functional food can be prepared by molding a mixture of the active ingredient of the present disclosure and excipients, binders, disintegrants, and the like, according to a known method. If necessary, it may be coated with white sugars or other coating aids, or the surface thereof may be coated with a material such as starch and talc.

The granule-type health functional food can be prepared by a conventionally known method in which a mixture of the active ingredient of the present disclosure and excipients, binders, disintegrants, and so one is formed into granules. If necessary, fragrance agents, flavoring agents, etc. can be added.

Particularly in light of the general features of natural extracts, when administered, the composition of the present disclosure is considered to cause less adverse effects, compared to synthetic medicaments. In fact, the composition was found to have no influences on the body as measured by standard toxicity assays.

Another aspect of the present disclosure pertains to a method for preventing or treating hair loss, the method comprising administering a composition comprising an Aucklandia lappa Decne. extract as an active ingredient to a subject in need thereof.

Another aspect of the present disclosure pertains to a method for alleviating or inhibiting hair loss, the method comprising administering a composition comprising an Aucklandia lappa Decne. extract as an active ingredient to a subject in need thereof.

Another aspect of the present disclosure pertains to a method for promoting hair regrowth, the method comprising administering a composition comprising an Aucklandia lappa Decne. extract as an active ingredient to a subject in need thereof.

Hereinafter, the present disclosure will be described in detail with reference to exemplary embodiments. However, these exemplary embodiments are for specifically illustrating the present disclosure and the scope of the present disclosure is not limited to these exemplary embodiments.

### PREPARATION EXAMPLE 1: PREPARATION OF WATER EXTRACT OF AUCKLANDIA LAPPA

To 100 g of Aucklandia lappa roots, the 10-fold volume 1.0 L of water was added, followed by extraction at 100° C. for 2 hours. Thereafter, the extract was concentrated in a vacuum and lyophilized. The amount of the powder thus obtained after drying is given in Table 1, below.

TABLE 1

| | | Amount of Extract |
|---|---|---|
| Sample | Solvent used | Powder (g) |
| Example 1 | Water | 25.1 |

### PREPARATION EXAMPLE 2: PREPARATION OF METHANOL EXTRACT OF AUCKLANDIA LAPPA

To 100 g of Aucklandia lappa roots, the 10-fold volume 1.0 L of methanol (100%) or a mixture of water and methanol (30, 50, or 70%) was added, followed by extraction at 100° C. for 2 hours. Thereafter, the extract was concentrated in a vacuum and lyophilized. The amounts of the powder thus obtained after drying are given in Table 2, below.

TABLE 2

| | | Amount of Extract |
|---|---|---|
| Sample | Solvent used | Powder (g) |
| Example 2 | Methanol 30% | 22.5 |
| Example 3 | Methanol 50% | 18.8 |
| Example 4 | Methanol 70% | 15.3 |
| Example 5 | Methanol 100% | 11.5 |

### PREPARATION EXAMPLE 3: PREPARATION OF ETHANOL EXTRACT OF AUCKLANDIA LAPPA

To 100 g of Aucklandia lappa roots, the 10-fold volume 1.0 L of ethanol (100%) or a mixture of water and ethanol (30, 50, or 70%) was added, followed by extraction at 100° C. for 2 hours. Thereafter, the extract was concentrated in a vacuum and lyophilized. The amounts of the powder thus obtained after drying are given in Table 3, below.

TABLE 3

| | | Amount of Extract |
|---|---|---|
| Sample | Solvent used | Powder (g) |
| Example 6 | Ethanol 30% | 23.1 |
| Example 7 | Ethanol 50% | 20.5 |
| Example 8 | Ethanol 70% | 17.3 |
| Example 9 | Ethanol 100% | 15.4 |

### PREPARATION EXAMPLE 4: PREPARATION OF ISOPROPANOL EXTRACT OF AUCKLANDIA LAPPA

To 100 g of Aucklandia lappa roots, the 10-fold volume 1.0 L of isopropanol (100%) or a mixture of water and isopropanol (30, 50, or 70%) was added, followed by extraction at 100° C. for 2 hours. Thereafter, the extract was concentrated in a vacuum and lyophilized. The amounts of the powder thus obtained after drying are given in Table 4, below.

TABLE 4

| | | Amount of Extract |
|---|---|---|
| Sample | Solvent used | Powder (g) |
| Example 10 | Isopropanol 30% | 22.4 |
| Example 11 | Isopropanol 50% | 19.7 |
| Example 12 | Isopropanol 70% | 16.5 |
| Example 13 | Isopropanol 100% | 14.1 |

### PREPARATION EXAMPLE 5: PREPARATION OF BUTANOL EXTRACT OF AUCKLANDIA LAPPA

To 100 g of Aucklandia lappa roots, the 10-fold volume 1.0 L of isopropanol (100%) or a mixture of water and isopropanol (30, 50, or 70%) was added, followed by extraction at 100° C. for 2 hours. Thereafter, the extract was concentrated in a vacuum and lyophilized. The amounts of the powder thus obtained after drying are given in Table 5, below.

TABLE 5

| | | Amount of Extract |
|---|---|---|
| Sample | Solvent used | Powder (g) |
| Example 14 | Butanol 30% | 20.9 |
| Example 15 | Butanol 50% | 17.8 |
| Example 16 | Butanol 70% | 15.1 |
| Example 17 | Butanol 100% | 12.2 |

### EXPERIMENTAL EXAMPLE 1: ASSAY FOR HEPATOTOXICITY AND NEPHROTOXICITY

Among the extracts prepared in the Preparation Examples, Examples 1 (extracted with 100% water), Example 2 (extracted with 30% methanol), and Example 6 (extracted with 30% ethanol) were selected due to their high yield and were assayed for hepatotoxicity and nephrotoxicity. Examples 1, 2, and 6 used for the assay were all administered in a dry powder form.

1-1. Drug Treatment and Construction of Tissue Slice

In order to examine in vivo toxicity of the Aucklandia lappa Decne. extracts, animal experiments were performed as follows. C57BL/6 mice at six weeks of age were purchased and acclimated to a breeding room for two weeks in a condition of about 22° C., a relative humidity of about 50%, and 12-hour light/dark cycles. Thereafter, the mice were divided into five groups of six heads which were non-treated for a control and orally administered Examples 1, 2, and 3, and Pansidil (medicinal yeast 100 mg, keratin 20 mg, thiamine nitrate mg, calcium pantothenate 60 mg, L-cysteine 20 mg, para-aminobenzoic acid 20 mg, animal-derived keratin), respectively (Table 6).

TABLE 6

| Group | Drug |
|---|---|
| Control | Non-treated |
| Example 1 | Example 1 200 mg/kg orally administered |
| Example 2 | Example 2 200 mg/kg orally administered |
| Example 6 | Example 6 200 mg/kg orally administered |
| Pansidil | Pansidil 200 mg/kg orally administered |

Following acclimation, the mice were orally administered the corresponding drugs at a dose of 200 mg/kg once a day for sex weeks. After completion of the oral administration, the mice were sacrificed to excise livers and kidneys. These organs were prepared into paraffin blocks, sectioned into slices, and stored before use.

1-2. Assay for Hepatotoxicity and Nephrotoxicity

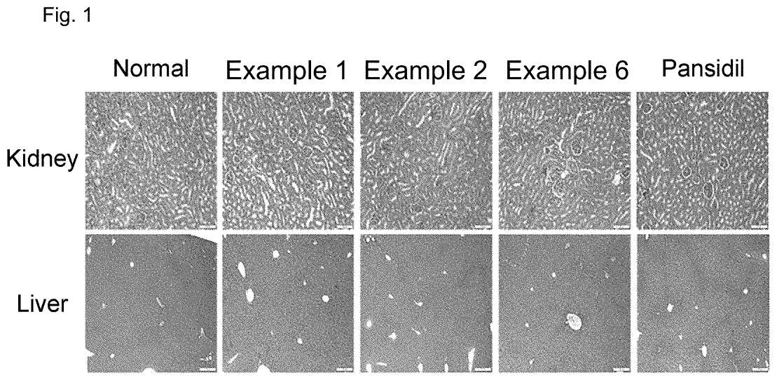

The tissue slices were deparaffinized and stained with hematoxylin & eosin, followed by observation and comparison of tissue under a microscope. The results are shown in FIG. 1.

As shown in FIG. 1, no tissue injury was observed in the livers and kidneys upon the oral administration of Examples 1, 2, and 6, and Pansidil, compared to the control. The liver and the kidney are organs that are most influenced by a drug injected into the body. However, oral administration of Examples 1, 2, and 6 was found to cause no toxicity in the livers and kidneys of the mice.

### EXPERIMENTAL EXAMPLE 2: ASSAY FOR HAIR GROWTH

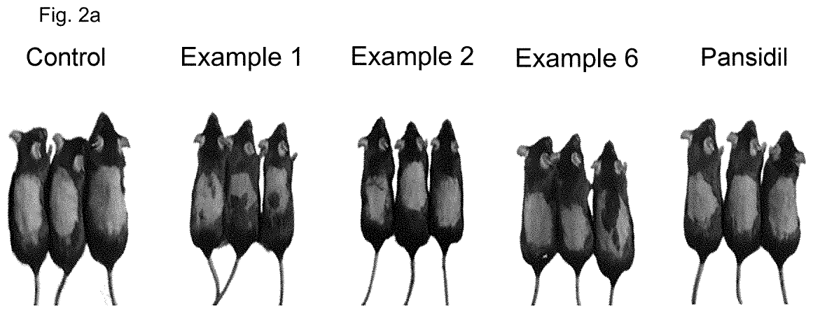

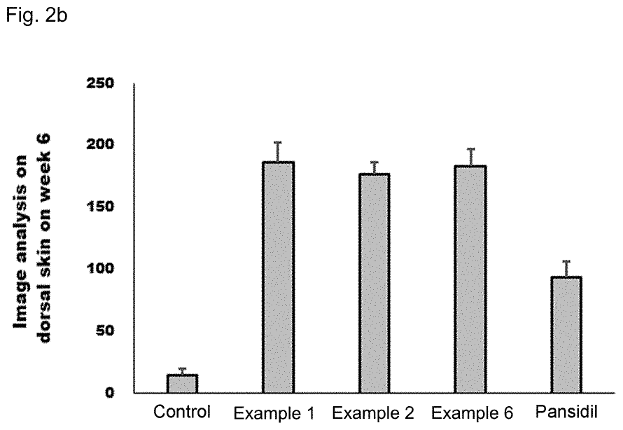

In order to examine the ability of the scalp composition of the present disclosure to promote hair regrowth, in vivo animal experiments were performed with mice. C57BL/6 mice 40 to 45 weeks old were purchased and acclimated to a breeding room for 2 weeks in the condition of about 22° C., relative humidity of about 50%, and 12-hours light/dark cycles. Thereafter, the mice had hairs shaved on their back. The shaved mice were divided into five groups of six heads. Of them, the four groups were orally administered Examples 1, 2, and 6 and Pansidil, respectively, at a dose of 200 mg/kg once a day for six weeks. Thereafter, hair growth states on the shaved areas were examined with the naked eye. The results are given in FIG. 2A. Proportions of the areas on which hairs had newly grown in the shaved total areas were quantitated and the results are given in FIG. 2B.

As shown in FIGS. 2A and 2B, the oral administration of Examples 1, 2, and 6 increased hair growth rates, compared to the non-treated control. In particular, the highest hair growth effect was obtained with Example 1, which was the water extract. In addition, Examples, 1, 2, and 6 were observed to exhibit higher hair growth effects, compared to Pansidil, which is a commercially available agent for hair loss.

### EXPERIMENTAL EXAMPLE 3: HAIR FOLLICLE OBSERVATION BY STAINING

3-1. Construction of Dermal Tissue Slice

In order to histologically observe numerical changes of hair follicles in the skin, mice were sacrificed by cervical dislocation after completion of the assay. Incisions were made in parallel to the spine line on the hair grown areas of the dorsal skin, followed by fixation with 4% paraformaldehyde. Thereafter, the fixed samples were sectioned into 7-μm thick slices on a cryostat.

3-2. Hematoxylin & Eosin Staining

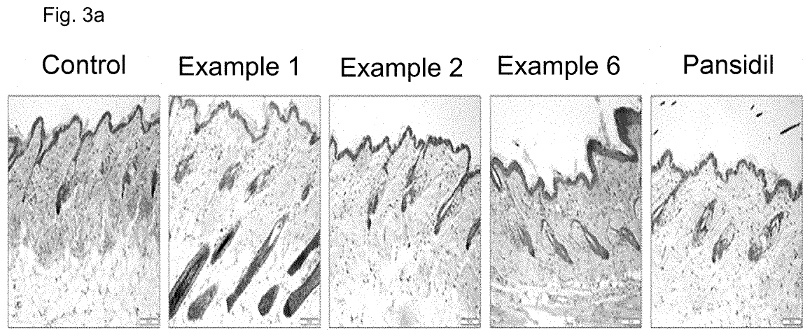

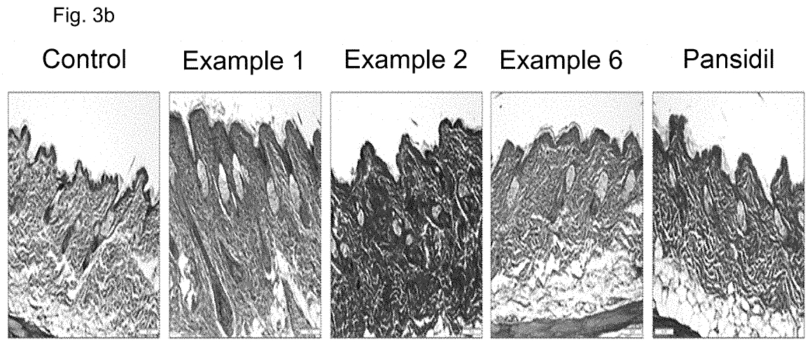

The skin tissue slices were deparaffinized and stained with hematoxylin & eosin. Then, the tissues were observed and compared under a microscope, and the results are given in FIG. 3A.

As shown in FIG. 3A, the groups to which Examples 1, 2, and 6 were administered were observed to have more hair follicles formed and better sebaceous gland developed, compared to the control. In particular, a better effect was obtained by Example 1, compared to Pansidil, which is a commercially available therapeutic agent for hair loss.

3-3. Masson Trichrome Staining

The dermal tissue slices were deparaffinized and stained using Masson's trichrome. Then, the tissues were observed and compared under a microscope, and the results are given in FIG. 3B.

As shown in FIG. 3B, collagen fibers of the skin tissues were well developed and uniformly distributed in the groups of Examples 1, 2, and 6, compared to the control.

As described hitherto, the present disclosure pertains to a composition comprising an Aucklandia lappa Decne. extract, especially a crude solvent extract from Aucklandia lappa as an active ingredient for alleviation, inhibition, prevention, or treatment of hair loss, and a preparation method therefor. The Aucklandia lappa Decne. extract according to the present disclosure can be used in a pharmaceutical or food composition useful for alleviating, inhibiting, preventing, and treating hair loss.

What is claimed is:

1. A method for prevention or treatment of hair loss, the method comprising:
administering a composition comprising an Aucklandia lappa Decne. extract as an active ingredient to a subject in need thereof.

2. The method of claim 1, wherein the Aucklandia lappa is an at least one entity selected from the group consisting of roots, stems, and leaves thereof.

3. The method of claim 1, wherein the Aucklandia lappa Decne. extract is obtained using at least one solvent selected from the group consisting of water, a straight or branched alcohol of 1 to 4 carbon atoms, acetone, ethyl acetate, butyl acetate, 1,3-butylene glycol, hexane, and diethyl ether.

4. A method for alleviation or inhibition of hair loss, the method comprising:
administering a composition comprising an Aucklandia lappa Decne. extract as an active ingredient to a subject in need thereof.

5. The method of claim 4, wherein the Aucklandia lappa is an at least one entity selected from the group consisting of roots, stems, and leaves thereof.

6. The method of claim 4, wherein the Aucklandia lappa Decne. extract is obtained using at least one solvent selected from the group consisting of water, a straight or branched alcohol of 1 to 4 carbon atoms, acetone, ethyl acetate, butyl acetate, 1,3-butylene glycol, hexane, and diethyl ether.

7. A method for promotion of hair regrowth, the method comprising:
administering a composition comprising an Aucklandia lappa Decne. extract as an active ingredient to a subject in need thereof.

8. The method of claim 7, wherein the Aucklandia lappa is an at least one entity selected from the group consisting of roots, stems, and leaves thereof.

9. The method of claim 8, wherein the Aucklandia lappa Decne. extract is obtained using at least one solvent selected from the group consisting of water, a straight or branched alcohol of 1 to 4 carbon atoms, acetone, ethyl acetate, butyl acetate, 1,3-butylene glycol, hexane, and diethyl ether.
